(19) United States
(12) Patent Application Publication
(10) Pub. No.: US 2020/0105419 A1
(43) Pub. Date: Apr. 2, 2020

(54) DISEASE DIAGNOSIS USING LITERATURE SEARCH

(71) Applicant: codiag AG, Kusnacht (CH)

(72) Inventors: Carsten Eickhoff, Providence, RI (US); Kai Habighorst, Küsnacht (CH); Floran Gmehlin, Zürich (CH)

(21) Appl. No.: 16/146,855

(22) Filed: Sep. 28, 2018

(51) Int. Cl. G16H 50/50; G06F 17/30; G16H 10/60; G16H 50/20; G06N 5/02

(52) U.S. Cl. CPC G16H 50/50; G06F 17/30867; G06N 5/02; G16H 10/60; G16H 50/20; G06F 17/3053

(57) ABSTRACT

Technology for predicting potential disease diagnoses of patients is disclosed. In an example, data associated with a patient is accessed. The data is divided into one or more queries. Each of the one or more queries is associated with one or more keywords. For each of the one or more queries, a plurality of literatures based on the one or more keywords is generated. A plurality of terms extracted from each of the plurality of literatures for each of the one or more queries is merged into a combined list of terms. One or more potential diagnoses are provided based on the combined list of terms.

### TECHNICAL FIELD

Aspects and implementations of the present disclosure relate to electronic health records, and more specifically, to provide a list of possible disease diagnoses based on electronic health records using literature search.

### BACKGROUND

An electronic health record (EHR) is an electronic version of a patient's health record charts and information. An EHR can include any patient data, including patient's medical history, diagnoses, medications, treatment plans, immunization dates, allergies, laboratory and test results, imaging, doctor's office visit notes, medical family history, etc. Data in an EHR system can be manipulated and processed for further usage by other electronic systems.

### SUMMARY

The following presents a simplified summary of various aspects of this disclosure in order to provide a basic understanding of such aspects. This summary is not an extensive overview of all contemplated aspects, and is intended to neither identify key or critical elements nor delineate the scope of such aspects. Its purpose is to present some concepts of this disclosure in a simplified form as a prelude to the more detailed description that is presented later.

In an aspect of the present disclosure, a system and methods are disclosed for providing a list of disease diagnoses based on data associated with a patient using searching of literature. In one implementation, a method comprises accessing data associated with a patient, dividing the data into one or more queries, wherein each of the one or more queries is associated with one or more keywords, generating, for each of the one or more queries, a plurality of literatures based on the one or more keywords, merging a plurality of terms extracted from each of the plurality of literatures for each of the one or more queries into a combined list of terms, and providing one or more potential diagnoses based on the combined list of terms.

In one implementation, a system comprises a memory and a processing device coupled to the memory, where the processor is to receive one or more user input associated with a patient; divide the one or more user input into one or more queries, wherein each of the one or more queries is associated with one or more keywords; generate, for each of the one or more queries, a plurality of literatures based on the one or more keywords; merge a plurality of terms extracted from each of the plurality of literatures for each of the one or more queries into a combined list of terms; and provide one or more potential diagnoses based on the combined list of terms.

In one implementation, a non-transitory computer readable storage medium encoding instructions thereon that, in response to execution by one or more processing devices, cause the processing device to perform operations comprising: accessing a health record associated with a patient; dividing the health record into one or more queries, wherein each of the one or more queries is associated with one or more keywords; generating, for each of the one or more queries, a plurality of literatures based on the one or more keywords; merging a plurality of terms extracted from each of the plurality of literatures for each of the one or more queries into a combined list of terms; and providing one or more potential diagnoses based on the combined list of terms.

In one implementation, a method comprises causing for display, by a processing device, a graphical user interface comprising: a first display component graphically depicting a health record associated with a patient, wherein the health record is divided into one or more sections, each of the one or more sections corresponding to a distinct medical episode; a second display component providing a plurality of literatures associated with the health record, wherein the plurality of literatures is generated based on one or more keywords associated with the health record; and a third display component providing one or more potential diagnoses based on terms extracted from each of the plurality of literatures associated with the health record.

Further, computing devices for performing the operations of the above described methods and the various implementations described herein are disclosed. Computer-readable media that store instructions for performing operations associated with the above described methods and the various implementations described herein are also disclosed.

### BRIEF DESCRIPTION OF THE DRAWINGS

Aspects and implementations of the present disclosure will be understood more fully from the detailed description given below and from the accompanying drawings of various aspects and implementations of the disclosure, which, however, should not be taken to limit the disclosure to the specific aspects or implementations, but are for explanation and understanding only.

FIG. 1 depicts an illustrative computer system architecture, in accordance with one or more aspects of the present disclosure.

FIG. 2 depicts a flow diagram of one example of a method for providing potential disease diagnoses, in accordance with one or more aspects of the present disclosure.

FIG. 3 depicts a system flow diagram for providing potential disease diagnoses, in accordance with one or more aspects of the present disclosure.

FIG. 4 depicts an example of term fusion, in accordance with one or more aspects of the present disclosure.

FIG. 5 depicts an example of a graphical user interface (GUI) of a disease diagnosis system, in accordance with one or more aspects of the disclosure.

FIG. 6 depicts an example of a graphical user interface (GUI) of a disease diagnosis system depicting performance statistics, in accordance with one or more aspects of the disclosure.

FIG. 7 depicts an example of a graphical user interface (GUI) of a disease diagnosis system depicting exclusion of an episode, in accordance with one or more aspects of the disclosure.

FIG. 8 depicts an example of a graphical user interface (GUI) of a disease diagnosis system depicting feedback providing mechanism, in accordance with one or more aspects of the disclosure.

FIG. 9 depicts a block diagram of an example computer system operating in accordance with one or more aspects of the disclosure.

### DETAILED DESCRIPTION

Data collected for and used in an electronic health record (EHR) system can be used in various ways to provide computer generated digital solutions in health care fields for patient care and clinical support. One of the uses of EHR systems can be in diagnosing diseases based on EHR data. EHR data can include structured data as well as free-form textual data. In conventional systems, clinical decision support systems are used to assist medical professionals in evaluating symptoms and making correct and timely decisions, aided by EHR data. These systems typically rely on identifying relevant information and conducting inferences on the basis of the relevant information. For example, these systems may use an EHR for a patient and provide a diagnosis or a list of diagnoses based on the EHR of the patient.

Many diagnosis systems generally rely on classifying EHR data based on historic patient data and classes of known diseases. For example, using historic patient data, patients with a particular symptom or set of symptoms may have been diagnosed with a particular disease. Given a new patient's EHR data, a system may provide a prediction of likelihood of the new patient having the particular disease based on the historic data. In doing so, machine learning, or deep learning, methodologies can be used to classify and predict disease diagnosis. For example, neural network learning using auto-encoders with EHR data has been used to predict disease diagnosis. In order for machine learning systems to predict an outcome, the machine learning system needs to be trained using historical data and categorization of the outcomes as training data for the machine learning system. However, there are various challenges in applying machine learning in disease diagnosis.

A reliable prediction using machine learning is possible with a large number of training data for each disease to be diagnosed. Healthcare related data tends to be sensitive and hard to collect. There may not be enough sample data available for use as training data for each and every existing disease. Specifically, the scarcity of the training data is acute for rare and undiagnosed diseases. In addition, a vast number of potential diagnostic classes need to be considered in order to classify the EHR data for disease diagnosis, adding complexity to the systems. For example, as many as twelve thousand disease classes have been known to exist in some systems. Classifying diseases using such a large number of potential diagnostic classes causes many technical problems. The challenges lead to narrowing down the scope of the diseases that can be diagnosed using these machine learning systems, leaving a vast landscape of diseases to be not recognized using these systems. As a result, disease diagnosis predictions using classification of diseases may be inaccurate and unreliable, in addition to being inefficient and expensive.

Aspects of the present disclosure address the above and other deficiencies by providing disease diagnosis mechanisms using a search mechanism based on data associated with a patient (e.g., her, user input, etc.) instead of a classification model. In one implementation, data (e.g., an EHR, user input, etc.) associated with a patient may be accessed. The data may be divided into one or more queries. For example, each query may represent a distinct medical episode, such as a patient encounter, a clinical visit, etc. Each of the queries may be associated with one or more keywords. A list of literatures may be generated based on the keywords for each of the queries. For example, the literature may be any type of document, including biomedical publications, articles, research papers, journal entries, textbooks, guidelines, or any other source of medical information. From each literature, multiple terms may be extracted. The terms may be merged into a combined list of terms. The combined list of terms may be used to identify and provide one or more potential disease diagnoses.

In some implementation, a graphical user interface (GUI) to present the various pieces of a disease diagnosis system may be provided for display on a computer system. The GUI may include a display component for depicting a health record (e.g., an EHR) associated with a patient. The health record may be divided into one or more sections. Each section may correspond to a distinct medical episode. The GUI may include a display component for providing a list of literatures associated with the health record. The list of literature may be generated based on one or more keywords associated with the health record. The GUI may include a display component for providing one or more potential diagnoses. The diagnoses may be generated based on terms extracted from the list of literatures associated with the health record. In some implementation, the health record may include data input by a user, an electronic health record (EHR), or a combination thereof.

Aspects of the present disclosure thus provide technology by which health records of patients can be used to predict disease diagnosis of patients. The technology allows for identification of diseases without the need for sample patient data. The technology allows for a patient's disease diagnosis to be predicted independent of other patients' historic data. The technology allows for disease diagnosis without the need to classify diseases into a number of classes and reduces complexity of disease diagnosis systems. As soon as a new disease is identified in a literature, the disease can be part of the search mechanism that serves the disclosed technology. The technology allows for greater scope of diseases to be diagnosed, including rare diseases. The technology provides for ease of access to disease diagnosis by providers and efficiency in computer resource. The technology allows for flexibility in terms of treating a patient by the patient's health care personnel. Accordingly, accuracy, reliability, and efficiency of disease diagnosis are improved using the aspects described in the present disclosure.

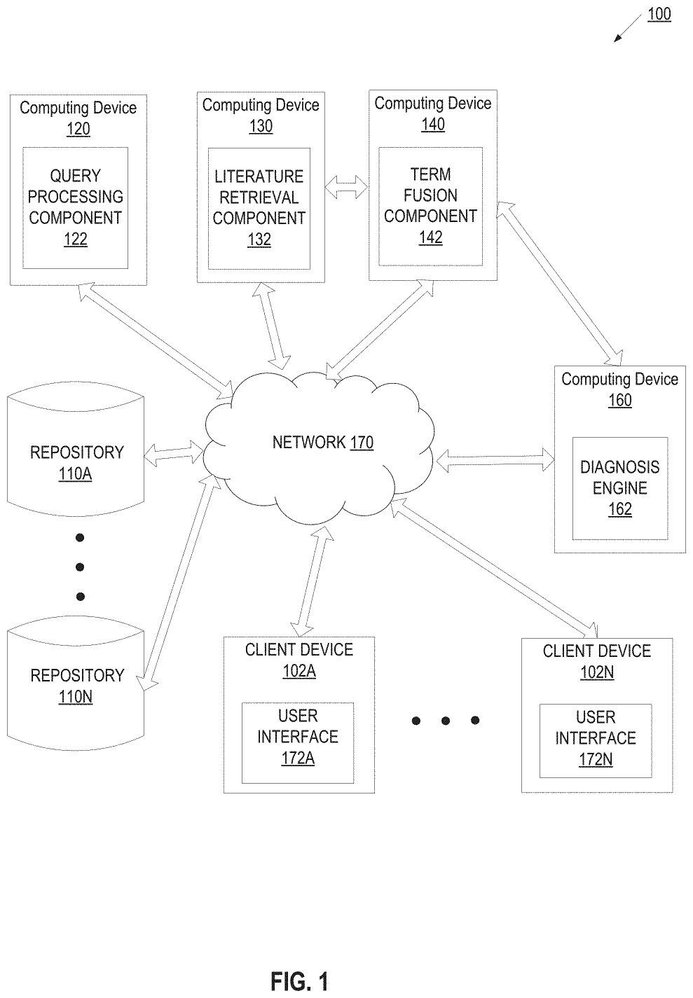

FIG. 1 illustrates an example system architecture 100, in accordance with one implementation of the present disclosure. The system architecture 100 includes one or more computing devices 120, 130, 140, 160, one or more repositories 110A through 110N, and client machines 102A-102N connected to a network 170. In some examples, computing devices 120-160 may be hosted using a cloud computing environment. Network 170 may be a public network (e.g., the Internet), a private network (e.g., a local area network (LAN) or wide area network (WAN)), or a combination thereof. The various computing devices may host components and modules to perform functionalities of the system 100. System 100 may include a query processing component 122, a literature retrieval component 132, a term fusion component 142, and a diagnosis engine 162.

The client devices 102A-102N may be personal computers (PCs), laptops, mobile phones, tablet computers, set top boxes, televisions, digital assistants or any other computing devices. The client machines 102A-102N may run an operating system (OS) that manages hardware and software of the client machines 102A-102N. In one implementation, the client machines 102A-102N may be used to monitor and predict health conditions of patients. Each of the client devices may include a user interface. Client devices 102A-102N may include user interfaces 172A-172N. User interfaces 172A-172N may include display components for depicting a health record associated with a patient, display components for providing a list of literatures, display components for presenting potential disease diagnoses, etc.

Computing device 120 may be a rackmount server, a router computer, a personal computer, a portable digital assistant, a mobile phone, a laptop computer, a tablet computer, a camera, a video camera, a netbook, a desktop computer, a media center, or any combination of the above. Computing device 120 may include an application specific integrated circuit (ASIC), a field programmable gate array (FPGA), a digital signal processor (DSP), other types of Integrated Circuits (IC), a distributed computing system, a cluster of machines, blockchain environment, or other compound combination of machines. Computing devices 130, 140, and 160 may be same as or comparable to computing device 120. In some examples, computing devices 120, 130, 140, and 160 may all be the same computing device.

Computing device 120 may include a query processing component 122 that is capable of processing a health record (e.g., an electronic health record including a patient's medical history, prior diagnoses, medications, treatment plans, immunization dates, allergies, laboratory and test results, imaging, doctor's office visit notes, physiological measurements, health attributes, conditions, procedures, etc.) from various data sources, including repositories 110A-N (e.g., using software agents, etc.). For example, query processing component 122 may connect to various types of Electronic Health Records (EHR) systems, hospital databases, physician data stores, patient portals, etc. Query processing component 122 may divide the health record into one or more queries. Each of the one or more queries may be associated with one or more keywords.

Repositories 110A-N may include persistent storage that is capable of storing a number of data types as well as data structures to tag, organize, and index health related data. Repositories 110A-N may be hosted by one or more storage devices, such as main memory, magnetic or optical storage based disks, tapes or hard drives, NAS, SAN, and so forth. In some implementations, repositories 110A-N may be network-attached file server, while in other implementations, repositories 110A-N may be other types of storage such as an object-oriented database, a graph based database, a document store, a key value store, a relational database, or combination thereof, that may be hosted by the computing device 120 or one or more different computing devices coupled to the computing device 120 via the network 170. The data stored in the repositories may include text data, numeric data, imaging data, structured data, documents, terms, etc. Repositories 110A-N may include repositories associated with various types of Electronic Health Records (EHR) systems, hospital databases, physician data stores, patient portals, various text documents such as surgical reports or imaging study reports, raw imaging data, genomic data, etc. In some implementations, repositories 110A-N may include repositories associated with various types of literature, including medical documents, journals, articles, research papers, textbooks, guidelines, reports, or any other source of medical information. In some examples, the repositories associated with the literatures may be directly accessed (e.g., live connection) by components of system architecture 100. In some examples, copies of the repositories or portions of the repositories associated with the literatures may be downloaded and stored as local copies within the system architecture 100. An example of a repository associated with literatures may include the Medical Literature Analysis and Retrieval System online (MEDLINE) providing bibliographic database of life sciences and biomedical information. In some implementations, repositories 110A-N may include repositories associated with various medical language libraries, including medical vocabularies, standards, classification tools, acronyms, etc. Some examples of medical language libraries may include the Unified Medical Language System (UMLS), QuickUMLS, the MetaMap developed by the National Library of Medicine (NLM), etc. In some examples, the repositories associated with the medical language libraries may be directly accessed (e.g., live connection) by components of system architecture 100. In some examples, copies of the repositories or portions of the repositories associated with the medical language libraries may be downloaded and stored as local copies within the system architecture 100.

Computing device 130 may include a literature retrieval component 132 that is capable of retrieving a plurality of literatures based on the one or more keywords associated with the queries obtained from query processing component 122. Computing device 140 may include a term fusion component 142 that is capable of extracting multiple terms from the literatures retrieved by literature retrieval component 132. Term fusion component 142 may fuse, or merge, the terms into a combined list of terms. The combined list of terms may be used to identify and provide one or more potential disease diagnoses. Computing device 160 may include a diagnosis engine 162 that is capable of providing provide one or more potential disease diagnoses based on the combined list of terms generated by the term fusion component 132.

It should be noted that in some other implementations, the functions of computing devices 120, 130, 140, and 160 may be provided by a fewer number of machines. For example, in some implementations two computing devices 130 and 140 may be integrated into a single computing device, while in some other implementations three computing devices 130, 140, and 160 may be integrated into a single computing device. In addition, in some implementations one or more of computing devices 120, 130, 140, and 160 may be integrated into a comprehensive disease diagnosis platform.

In general, functions described in one implementation as being performed by the comprehensive disease diagnosis platform, computing device 120, computing device 130, computing device 140, and/or computing device 160 can also be performed on the client machines 102A through 102N in other implementations, if appropriate. In addition, the functionality attributed to a particular component can be performed by different or multiple components operating together. The comprehensive disease diagnosis platform, computing device 120, computing device 130, computing device 140, and/or computing device 160 can also be accessed as a service provided to other systems or devices through appropriate application programming interfaces.

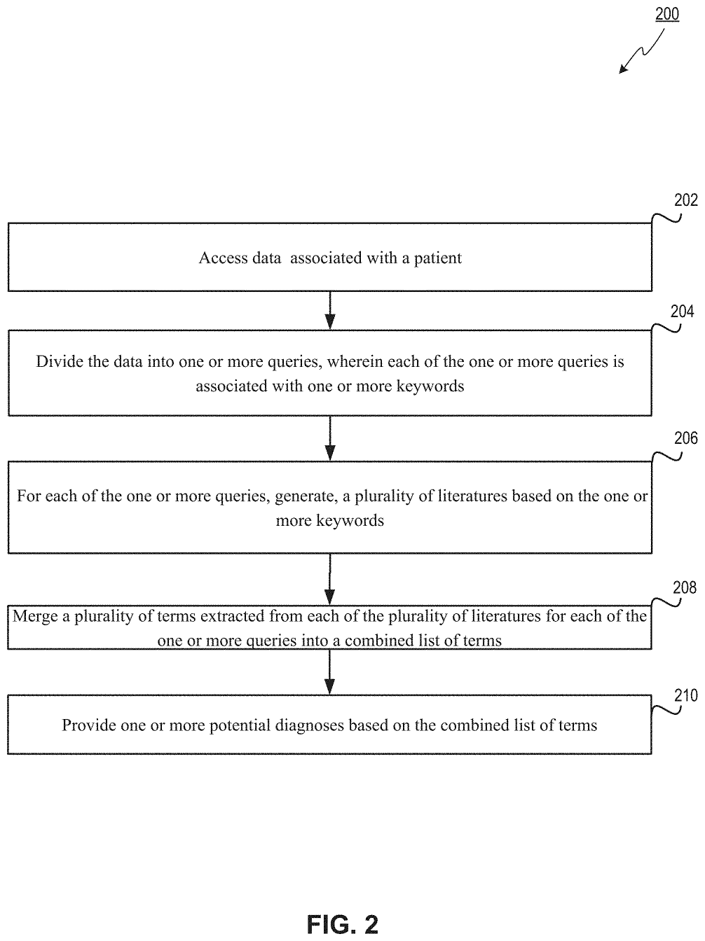

FIG. 2 depicts a flow diagram of one example of a method 200 for providing potential disease diagnoses, in accordance with one or more aspects of the present disclosure. The method is performed by processing logic that may comprise hardware (circuitry, dedicated logic, etc.), software (such as is run on a general purpose computer system or a dedicated machine), or a combination thereof. In one implementation, the method is performed by computer system 100 of FIG. 1, while in some other implementations, one or more blocks of FIG. 2 may be performed by one or more other machines not depicted in the figures. In some aspects, one or more blocks of FIG. 2 may be performed by various components depicted in FIG. 1.

For simplicity of explanation, methods are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events. Additionally, it should be appreciated that the methods disclosed in this specification are capable of being stored on an article of manufacture to facilitate transporting and transferring such methods to computing devices. The term article of manufacture, as used herein, is intended to encompass a computer program accessible from any computer-readable device or storage media.

Method 200 begins at block 202, where data associated with a patient is accessed. In some implementation, the data may include a health record, a user input, or a combination thereof. For example, a health record may include an electronic health record (EHR) including a patient's medical history, prior diagnoses, medications, treatment plans, immunization dates, allergies, laboratory and test results, imaging, doctor's office visit notes, physiological measurements, health attributes, conditions, procedures, etc. In one example, a health record for a patient can include all aggregate data associated with the patient, notes from multiple visits, etc. In another example, a health record may include a portion of the patient's aggregate health data. In some examples, a user input can include one or more terms or keywords input (e.g., entered) by a user. In an example, the user can input the terms or keywords using a graphical user interface. In another example, the user can input the terms or keyword using a system component, a batch database job, a script, etc. In some examples, the user can be a human user or a system user.

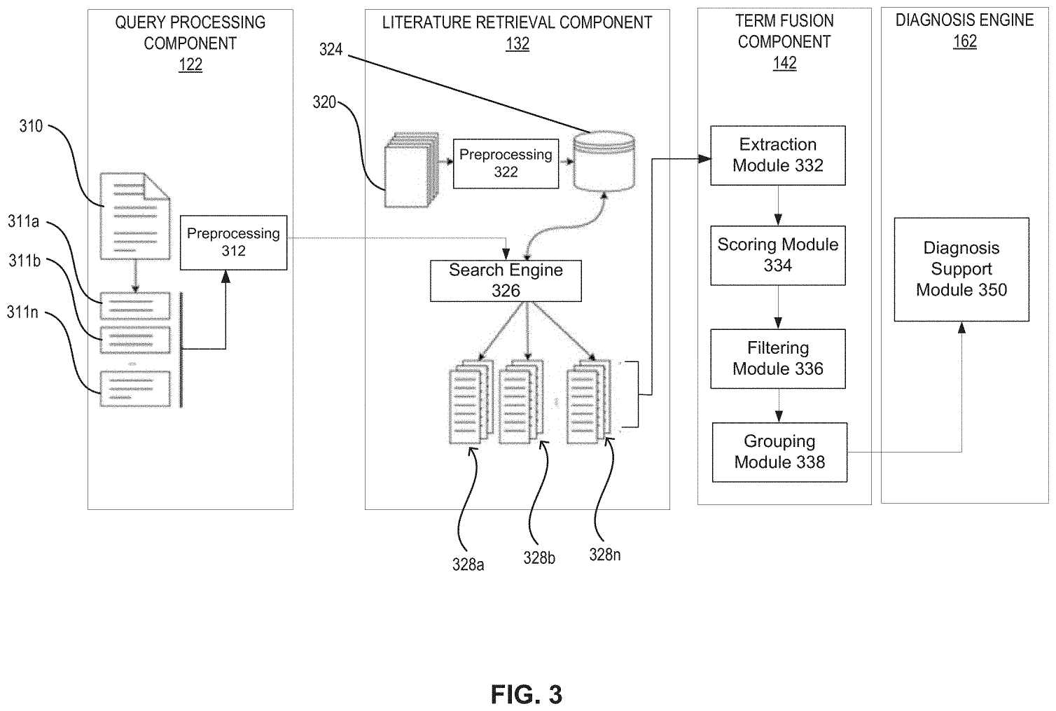

For example, FIG. 3 depicts an example system flow diagram for providing potential disease diagnoses. In the example of FIG. 3, query processing component 122 of FIG. 1 is shown as accessing health record 310. However, in other examples, other components depicted in FIG. 1 may be used to perform block 202.

Referring back to FIG. 2, at block 204, the data may be divided into one or more queries. Typically, an EHR includes a lengthy and topically diverse set of data. As such, a query may be obtained by dividing an EHR into a coherent clinical episode. Each of the one or more queries may represent a distinct medical episode, such as a patient encounter, a clinical visit, etc. For example, for an EHR that includes multiple clinical visits, each visit may be categorized as a distinct query. The content of the query may consist of notes and other data from each individual visit. In an example, during a single medical episode, such as a clinical visit, a patient's condition may be investigated and documented by a clinician in the form of a clinical note. The clinician may enter the note (e.g., text, lab results, etc.) into an EHR system during the visit. The EHR system may assign an identifier for each note. As notes are entered into the EHR system, the notes may be chronologically ordered. The note may be appended to the previous note (e.g., from a previous visit), which make up the overall EHR for the patient. When dividing the EHR into queries, each note having a different identifier may be identified as a distinct query. In another example, machine learning models can be used to divide an EHR into queries, where a machine learning model learns from training data about how previous EHRs have been divided into queries, and apply it to a patient's current EHR to divide the EHR into queries.

In the example of FIG. 3, patient record 310 is divided into queries 311a, 311b, through 311n. The queries may be represented as a set Q={Q₁, Q₂, . . . , Qₙ}, where Q₁ corresponds to query 311a, Q₂ corresponds to query 311b, Qₙ corresponds to query 311n, etc. An illustrative example of a patient suffering from celiac disease is provided below. The clinical episodes are ordered temporally and each consecutive consultation corresponding to each query reveals additional information as compared to the previous set of queries. The queries Q₁, Q₂ and Q₃ in the example are as follows:

Q₁: A 13 year old female living in a remote rural area came to our clinic with an 8 year history of deformities in the extremities [ . . . ] developed recurrent fractures in her legs and arms after minor falls. [ . . . ] There were no gastrointestinal symptoms of abdominal pain or diarrhea. She had been diagnosed with rickets and iron deficiency anemia [ . . . ] and had received Vitamin D and iron supplements many times without improvement. [ . . . ] The patient was pale. She had severe bowing of her arms and legs.

Q₂: X-rays of her upper and lower limbs showed diffuse osteopenia and bowing of both legs and forearms with blurring of the metaphyseal lines. It also showed dense transverse lines in tibia and ulna suggestive of looser's zones indicative of severe rickets.

Q₃: Anti-endomysial antibodies titer was 80 (normal is negative), anti-tissue transglutaminase IgA was positive 75 U/ml (normal below 2.5 U/ml) and anti-tissue transglutaminase IgG was negative. [ . . . ] The duodenum showed scalloping and fissuring of the small bowel. The histopathology report of the small intestine showed severe villous atrophy grade IV with crypt hyperplasia. [ . . . ] Total villous atrophy with completely flat mucosa and increased intraepithelial lymphocytes.

Each of the one or more queries may be associated with one or more keywords (e.g., words, terms, acronyms, etc.). In some implementations, a preprocessing operation may be performed on each of the queries. The preprocessing operation may be performed in order to filter out keywords in a query that do not add value to the diagnosis prediction process and to remove an uninformative keyword from the one or more keywords. For example, from the content of a query, keywords such as stop words, uninformative part-of-speech tags such as verbs, determiners, adpositions, coordinating conjunctions, and punctuations can be removed. The remaining context bearing keywords may be kept as part of the query. In some implementations, the system can customize the type of keywords to include and the type of keywords to exclude as part of the query preprocessing operation, such that a user may have the option to customize the query preprocessing operation. In the example of FIG. 3, an operation preprocessing 312 is performed on the queries 311a-311n.

At block 206, a plurality of literatures may be generated for each one of the one or more queries. For example, the literature may be any type of document, including medical documents, biomedical publications, articles, research papers, journal entries, scholarly reports, expert literatures, etc.

The literatures may be generated using a collection of literatures retrieved from various sources. In some examples, the collection of literatures can be retrieved from multiple sources. In some examples, the collection of literatures can be retrieved from a central literature database. An example of a central database of literatures may include the publicly available source Medical Literature Analysis and Retrieval System online (MEDLINE) providing bibliographic database of life sciences and biomedical information. In some examples, the literatures may be directly accessed from the literature source. In some examples, the literatures or portions of the literatures may be copied or downloaded to a local database accessible to the diagnosis system. In some examples, a combination of direct access and local copies may be used.

In some implementations, the collection of the literatures may be pre-processed prior to further use by the system. For example, in an example where the collection of literatures is downloaded to a local database of the system, the collection may be downloaded as one record of a series of records that include multiple documents. Once the collection is downloaded, the system may split the record(s) into individual documents (e.g., literature) by performing a preprocessing operation. In some implementations, the collection of literatures may be indexed. The indexing is used to break up the data into terms that can be searched. The indexed terms may be associated with each of the respective individual documents.

In the example of FIG. 3, literature retrieval component 132 retrieves a collection of literatures 320. A preprocessing 322 operation is performed on the collection of literatures 320 to split the collection of literatures into individual literatures. The literatures are additionally indexed into multiple terms associated with each individual literature and stored into an index database 324. Search engine 326 uses the index database 324 to perform searches on the collection of literatures 320.

The plurality of literatures may be generated based on the one or more keywords associated with the one or more queries. For each query of the one or more queries, the plurality of literatures may be generated using a search engine. The search engine may be used to search the collection of literatures using the one or more keywords associated with each query. Thus, the search engine can provide a list of literatures corresponding to each of the queries based on the one or more keywords and an index database of terms related to the literatures. In the example of FIG. 3, queries 311a-311n are sent to the search engine 326. Search engine 326 uses the one or more keywords associated with each query (e.g., 311a) to search the collection of literatures 320, using the index database 324 containing indexed terms from the collection of literatures 320, to generate a list of literatures for each of the queries. Search engine 326 generates a plurality of literatures 328a for query 311a, a plurality of literatures 328b for query 311b, a plurality of literatures 328n for query 311n, etc.

In some implementations, literatures within each of the plurality of literatures corresponding to each query may be ranked. A rank for each of the plurality of literatures may be calculated according to each of the one or more queries. The rank of each literature may be proportionate to the number of matches between terms of a literature and keywords of a query, such that, the larger the number of matches, the higher the rank of the literature within the plurality of literatures. That is, a literature within the plurality of literatures for a query may have a high rank if the literature matches a large number of terms as the keywords from the query. In some examples, the rank of the literature may be calculated using a relevance score associated with each of the literatures. In some examples, the relevance score may be calculated for each literature. In an example, the relevance score may be calculated based on the number of matches between the keywords for a query and terms of each literature. In an example, the higher the number of matches for a particular literature, the higher will be the relevance score assigned for that particular literature. In some examples, a Bayesian language model with Dirichlet priors may be used to rank the literatures.

In some implementations, the plurality of literatures may comprise a specified number of literatures. In some examples, the specified number may be a predefined number (e.g., 5 literatures, etc.). In some examples, the specified number may be dynamically selected based on relevant documents available for each of the queries. For example, the specified number may be dynamically selected based on the relevance score associated with the literature. The relevance score between two consecutively ranked literatures may be compared to identify a difference between the relevance scores. The specified number may be determined for each query based on the difference between the relevance score having the highest (e.g., largest) value. For example, comparing a list of consecutively ranked literatures and starting with the literature having the largest relevance score value, the point where the relevance score difference is highest between two consecutively ranked literatures can be selected as the cutoff point at which no more literatures may be included within the specified number of literatures. The cutoff point may include the literature with the larger value of the two relevance score values having the largest difference.

In an illustration for the ranking of the plurality of literatures, all documents d∈D may be ranked according to query Qᵢ to generate a ranking Lᵢ, for {i=1 to n}, where n is the total number of queries, d represents an individual document (e.g., literature), D represents a document collection consisting of the plurality of documents (e.g., literatures) generated for each query, Qᵢ represents the i^{th} query, and Lᵢ represents the resulting ranked list of documents corresponding to the i^{th} query. The query specific document ranking Lᵢ may have a length p (e.g., consisting of a p number of documents. Lᵢ may be represented as:

Lᵢ=argmaxₚ(P(Qᵢ,D)).

Where P(Qᵢ, D) represents the estimated probability of each of the documents in D being relevant to query Qᵢ.

The objective of selecting a value for the length p of the ranked list may be to keep the literatures with the highest relevance scores and to discard the less informative literatures. In some examples, when a distribution of the relevance scores of the plurality of literatures for a query is plotted on a linear graph starting with the largest (e.g., highest) value of the relevance score, a recurrent form of “L-shape” may be noticed. That is, the relevance score values of an initial set of literatures are significantly higher than the remainder of the distribution. The end portion of the distribution converges to meaningless values for the relevance score where the literatures are barely related to the keywords from the query. The point in the plot at which the distribution drops significantly may be the point where the relevance score difference is highest between two consecutively ranked literatures. This point can be selected as the cutoff point for selecting the length p (e.g., specified number of literatures), such that no more literatures may be included within the plurality of literatures after the cutoff point. Using the cutoff point, the literatures with high relevance score values can be kept within the plurality of literatures.

The point in the plot at which the distribution drops significantly (e.g., the cutoff point) can be query specific (e.g., the point can vary from one query to another query). In order to identify the cutoff point, the length p can be determined separately for each query. In some examples, the length p may be calculated based on the number of literatures at the “elbow” point (e.g., the cutoff point where relevance score value difference is largest) of the plot where the steepest chance in the curvature of the plot is located. The calculation can be reduced to finding the point p on the curve (e.g., plot) with the longest perpendicular distance d⊥(\vec{p}, \vec{b}) to the secant vector \vec{b} connecting the first and last document of result list Lᵢ. Accordingly, the point p can be calculated such that:

$${argmax}_{p} d ⊥ ( \vec{p} , \vec{b} ) = \vert \vec{p} - ( \vec{p} \cdot {\vec{b}}^{⋀} ) {\vec{b}}^{⋀} \vert$$

$${\vec{b}}^{⋀} = \frac{\vec{b}}{‖ \vec{b} ‖}$$

where(\vec{p}·\vec{b}\hat{ })\vec{b}\hat{ } is the orthogonal projection of vector \vec{p} onto vector \vec{b}.

Referring back to FIG. 2, at block 208, a plurality of terms for each query maybe merged into a combined list of terms. The plurality of terms may be extracted from each of the plurality of literatures. In some examples, the plurality of terms may be determined based on an overall score calculated for each of the plurality of terms. In some examples, the overall score may be calculated based on a term score indicating a term frequency-inverse document frequency for a particular term of the plurality of terms and the relevance score associated with a particular literature corresponding to the particular term. In some examples, the plurality of terms is determined by identifying, using a medical language library, a set of terms to remove (e.g., filtered) from an initial set of extracted terms from each of the plurality of literatures. In some examples, one or more synonymous terms of the plurality of terms may be grouped under a unique identifier corresponding to a potential diagnosis of the one or more potential diagnoses. In one example, as shown in FIG. 3, term fusion component 142 may perform operations of block 208. The term fusion component 142 may include an extraction module 332, a scoring module 334, a filtering module 336, and a grouping module 338.

Extraction module 332 may extract the plurality of terms from the plurality of literatures. The extracted terms may include textual content, including words, symbols, characters, acronyms, etc. from each of the plurality of literatures. In some implementations, a selected set of terms may be selected as the plurality of terms from all existing terms within a literature. In some examples, a document internal “tf-idf” (“term frequency-inverse document frequency”) terms may be identified, which represents terms that occur frequently locally (e.g, within the literature) but infrequently globally (e.g., across the collection of literatures). A tf-idf score of a term increases proportionally to the number of times a word appears in a document and is offset by the number of documents in the corpus that contain the word. Thus, high ranking tf-idf terms may correspond to terms that are meaningful for a particular disease as the terms appear more frequently within a particular literature but not common across all literatures. The system may set a threshold value for the tf-idf score such that terms with tf-idf score above the threshold value may be extracted for use as the plurality of terms.

In some implementations, additional processing of the extracted terms may be performed to obtain meaningful terms for the diagnosis process. For example, acronyms and synonyms may present a challenge when processing terms from the retrieved literatures. Acronyms and synonyms for a word may interfere with the downstream scoring of the terms and artificially cause discrepancies between the calculated score and actual score of a term. As such, acronyms and synonyms may be detected and processed to limit the effect of their existence within the literatures and to determine a more accurate calculation of the terms. For example, a literature with a high relevance score may contain a term “cd.” The term “cd” can be resolved as either “celiac disease” or “crohn's disease.” Depending on the interpretation selected, the predicted diagnoses may vary greatly. In order to disambiguate such an acronym, various medical language libraries may be used. The libraries may include medical vocabularies, standards, classification tools, acronyms, etc. A map of certified disease acronyms and their possible meanings may be extracted from one or more medical libraries. For example, a map for the acronym “cd” may be as follows:
‘cd’→[‘celiac disease’, ‘crohn disease’].

For each encountered acronym in each literature, corresponding article title or other designated portions may be checked to compare to the possible meanings of the encountered acronym according to the certified disease acronym to determine an interpretation for the acronym. If a match is found, the acronym may be replaced by its full form according to the map. For example, the title “Ulcerative jejunitis in a child with celiac disease,” which includes the words “celiac disease,” can be used to disambiguate the extracted term “cd” into “celiac disease.” In some examples, if none of the full forms present in the map for “cd” can be found in the title or a designated portion of the literature, the acronym may not be disambiguated and left as is.

In some implementations, the scoring module 334 may calculate an overall score for each term of the extracted terms. In some examples, the overall score may be calculated based on the tf-idf score for a particular extracted term and the relevance score of the literature containing the particular term. In some examples, the relevance scores may be combined in an additive manner, such as using a “CombSUM” method.

In an illustration for calculating the overall score for the term, the union of the η most highly-ranking tf-idf terms in each document din Lᵢ may be denoted as the set τ_{i,η} and expressed as:

τ_{i,η}=u_{d∈L}_{ᵢ} argmax_{η}tfidf(t,d).

For each termt ∈τ_{i,η}, its document-internal tf-idf scoretfidf(t, d) and the relevance score of the document d containing t may be computed. The higher the tf-idf score and the document relevance score, the higher the term's overall score will be.

The fusion scheme f, may be used to score terms in the following manner:

f(α,β,t)=Σᵢ₌₁ⁿ αtfidf(t,d)+βP(Qᵢ,d)

where α and β represent real-valued mixture weights and n is the total number of queries. In order to ensure comparability of query-specific relevance scores, raw scores for each query Qᵢ may be normalized.

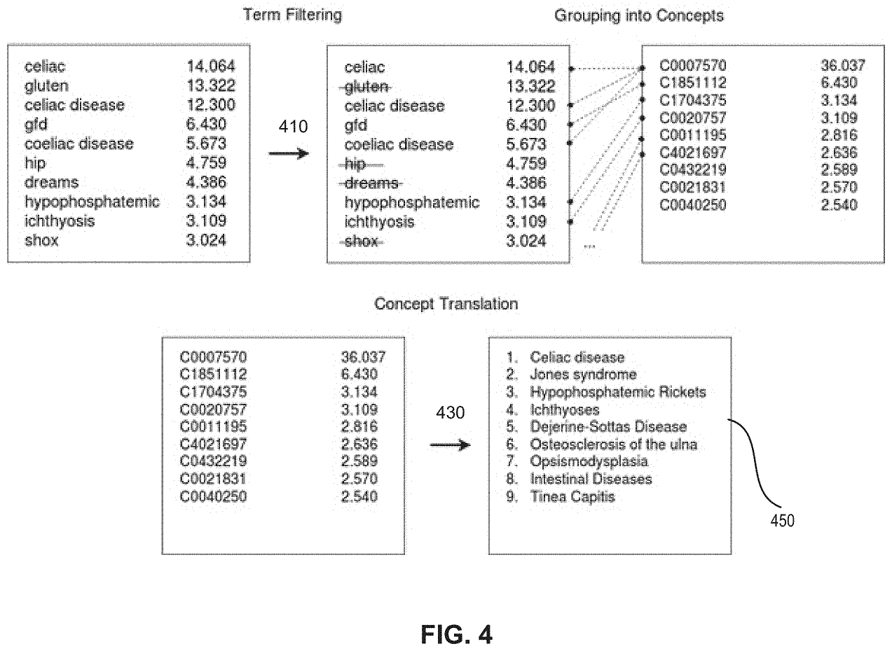

In some implementations, filtering module 336 may perform filtering operations on the plurality of terms. For example, some of the terms of the plurality of terms may contain little to no useful information for the disease diagnosis process. In some cases, these terms may indeed have a high tf-idf score, yet not be useful for the disease diagnosis process. For instance, terms like “Monday,” “dreams,” or “she” are not informative in the context of the application of disease diagnosis. These terms may be filtered out (e.g., removed) from the plurality of terms. In some examples, a medical language library (e.g., the UMLS) may be used to filter out terms that are not associated with a semantic type assigned to a term in the library that is useful for disease diagnosis. For example, for a given extracted term from a literature, corresponding semantic type of the given term may be retrieved from the library. If the semantic type is not “disease” or “syndrome” then the term may be filtered out of the plurality of terms. In the example of using the UMLS, if the semantic type does not belong to the type “[T047] Disease or Syndrome” then the term is filtered out. For example, FIG. 4 depicts an example of term fusion using term fusion component 142. In FIG. 4, arrow 410 shows an example of term filtering. Terms such as “gluten,” “hip,” “dreams,” “shox,” etc. have been filtered out, or removed, from the initial list of extracted terms, as these terms do not belong to the semantic type of disease or syndrome.

In some implementations, grouping module 338 may group synonymous terms of the plurality of terms together. That is, terms with similar meaning may be grouped together. The grouping can be done using unique identifiers, such that all terms with synonymous meanings are grouped under the same unique identifier. The identifier may correspond to an identifier in the particular medical language library used. For example, when the UMLS is used, the terms can be grouped under a Concept Unique Identifier (“CUI”). In an example, celiac disease can have different commonly used synonyms, such as, “Gluten Enteropathy,” “Non-Tropical Sprue,” or “Idiopathic Steatorrhea,” etc. Using UMLS, the terms can be grouped under the same concept, namely, “C00007570” which is the CUI of Celiac Disease.

In an implementation, the terms from each of the plurality of literatures for each of the queries may be merged together to form a combined list of terms. In some examples, after performing the term extraction, scoring, filtering, and grouping for the terms found in each list of literatures, the terms corresponding to all queries may be aggregated.

Referring back to FIG. 2, at block 210, one or more potential diagnoses based on the combined list of terms may be provided. In the example of FIG. 3, a diagnosis support module 350 of diagnosis engine 162 may perform the operation of block 210. In an example, after the terms are merged into a combined list of terms, a set of unique identifiers (e.g., CUIs) may be obtained under which the various terms of the combined list of terms are grouped. In some examples, if multiple terms fall under the same unique identifier (e.g., CUI), their individual scores (e.g., overall score calculated by the scoring module 336) may be combined to derive an overall score across all queries for each unique identifier. The diagnosis support module 350 may provide the list of one or more potential disease diagnoses based on the unique identifier under which terms have been grouped. As shown in FIG. 4, in some examples, a concept translation, as shown using arrow 430, may be performed. The concept translation is performed by finding the description associated with the unique identifier (e.g., CUI for UMLS) in the medical library, such as, finding the name of the disease for which the unique identifier stands for. As a result of the concept translation, the unique identifiers can be transformed into human readable disease diagnosis that can be provided as the one or more potential disease diagnoses. In some implementations, there may be a threshold value associated with the overall scores of the terms in the combined list of terms. In some examples, the diagnoses included in the one or more potential diagnoses may correspond to the unique identifiers having the overall scores above the threshold value. In the example of FIG. 4, the diagnosis support module 350 provides a list of potential disease diagnoses 450 based on the queries and corresponding terms. The list of diagnoses may be provided in an order of ranks calculated for each diagnosis based on the overall score corresponding to each grouping of the unique identifiers. A higher overall score may generate a higher rank. The system may calculate the ranks after each query is processed and before merging the results of the diagnosis.

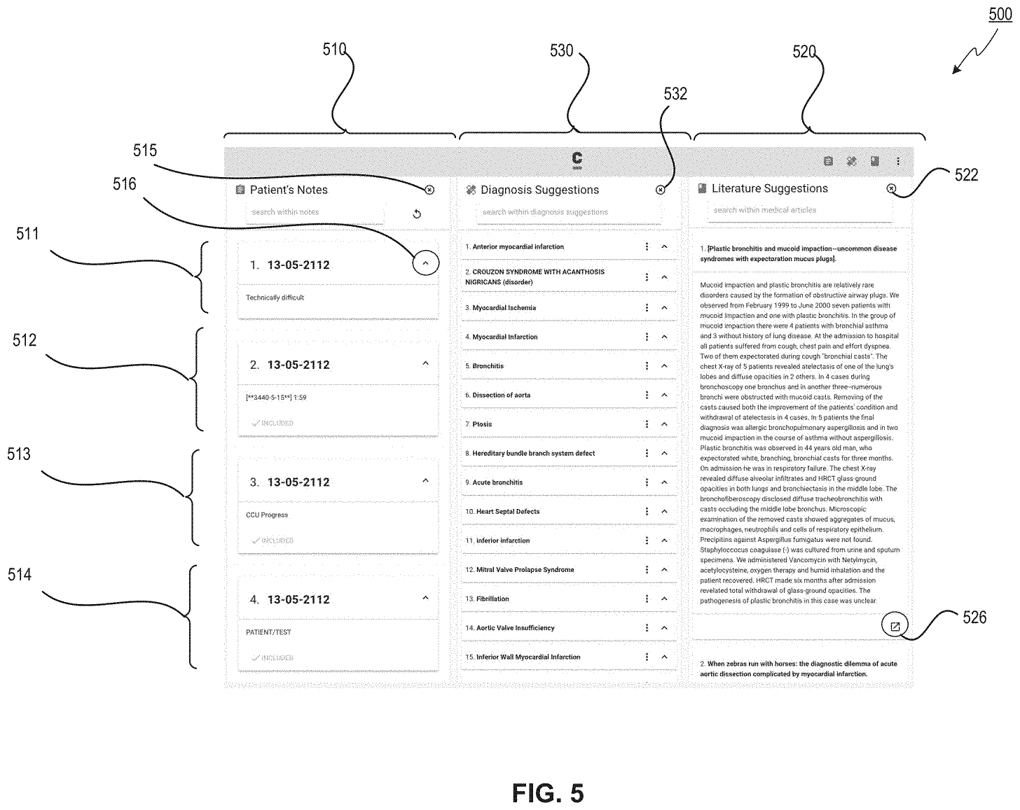

FIG. 5 depicts an example of a graphical user interface (GUI) 500 of a disease diagnosis system, in accordance with one or more aspects of the disclosure. A method may be performed to cause for display the graphical user interface. The GUI may include a first display component graphically depicting a health record associated with a patient, wherein the health record is divided into one or more sections, each of the one or more sections corresponding to a distinct medical episode; a second display component providing a plurality of literatures associated with the health record, wherein the plurality of literatures is generated based on one or more keywords associated with the health record; and a third display component providing one or more potential diagnoses based on terms extracted from each of the plurality of literatures associated with the health record. In some examples, the method may further comprise detecting a change in the health record. In some examples, the method may further comprise detecting a user selection to refresh the GUI or run the diagnosis process. In some examples, the method may further comprise receiving a user selection to include or exclude a section (e.g., corresponding to one or more queries) of the health record. In the above examples, responding to detecting a change, detecting a user selection to refresh the GUI or run the diagnosis process, or receiving a user selection to include/exclude a EHR section, the method may comprise updating the first display component to depict the changed, refreshed, or included/excluded health record section, respectively; updating the second display component to depict an updated plurality of literature associated with the changed, refreshed, or included/excluded health record section, respectively; and updating the third display component to provide an updated one or more potential diagnoses based on the changed health record, refreshed, or included/excluded health record section, respectively. In some implementation, the health record may include data input by a user, an electronic health record (EHR), or a combination thereof. For example, data input by a user (e.g., user input) can include one or more terms or keywords input (e.g., entered) by a user. In an example, the user can input the terms or keywords using the graphical user interface depicted in FIG. 5, or another, different graphical user interface. In another example, the user can input the terms or keyword using a system component, a batch database job, a script, etc. In some example, the user can be a human user or a system user.

In FIG. 5, the GUI 500 includes a first display component 510 depicting a health record (e.g., EHR) associated with a patient. The EHR is divided into one or more sections 511-514, which also correspond to one or more queries. Each of the one or more sections corresponding to a distinct medical episode. Button 515 may be clicked to show or hide the patient's EHR, alternatively. Button 516 may be clicked to expand or close the first clinical note corresponding to section 511. The GUI 500 includes a second display component 520 depicting a plurality of literatures associated with the health record. Button 522 may be clicked to show or hide, alternatively, the plurality of literatures. Button 526 may be clicked to open a link to the full text article for the second literature of the plurality of literatures. The GUI 500 includes a third display component 530 providing one or more potential diagnoses. Button 530 may be clicked to show or hide the diagnoses.

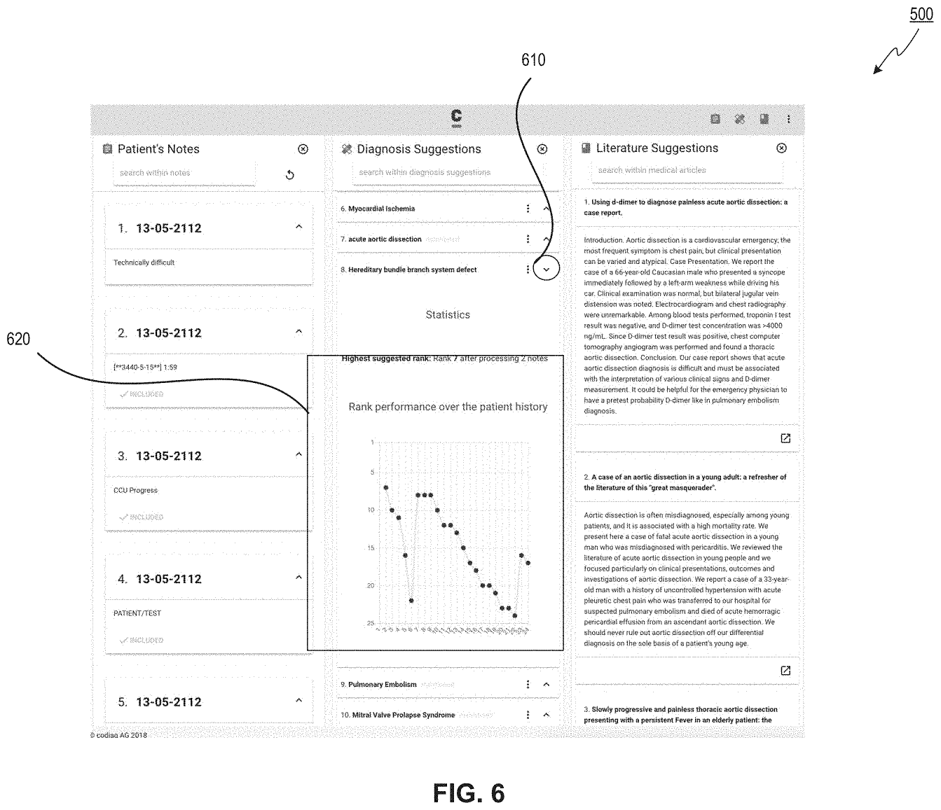

FIG. 6 depicts an example of a graphical user interface (GUI) 500 of a disease diagnosis system showing performance statistics, in accordance with one or more aspects of the disclosure. Arrow 610 may be clicked to show or hide more details for a specific diagnosis from the displayed list of potential diagnoses. The GUI 500 displays a performance statistics 620 associated with the particular diagnosis once the details for the diagnosis are shown. In the example shown, the patient has 24 notes (e.g., corresponding to 24 queries). The performance statistics 620 shows a graph of the rank calculated for the diagnosis after processing each of the notes (e.g., queries). The ranks for the diagnosis are provided on the Y axis and the notes are provided on the X axis.

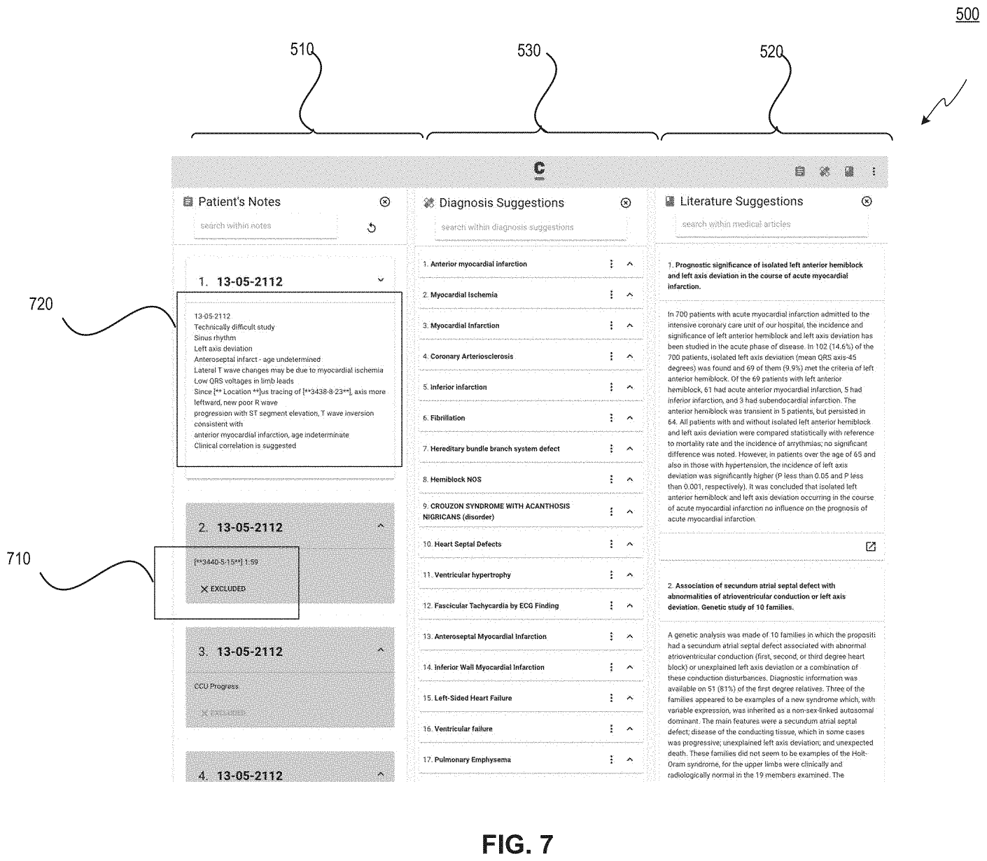

FIG. 7 depicts an example of a graphical user interface (GUI) 500 of a disease diagnosis system depicting exclusion of an episode, in accordance with one or more aspects of the disclosure. In the example, the first display component 510 displays a selection option 710 for a query (e.g., the second patient note here) which can be used to include or exclude a particular section (e.g., note, query, etc.) of an EHR for the patient from the diagnosis process. In the example, the display component 510 provides an indication that the particular section (e.g., the second patient note) has been “excluded” from the diagnosis process. As a result of excluding the particular section, the second display component 520 is updated with an updated plurality of literatures associated with the excluded health record section, and the third display component 530 is updated with an updated one or more potential diagnoses based on the excluded health record. Additionally, first display component 510 is depicted as showing details 720 of the first patient note after the note has been expanded.

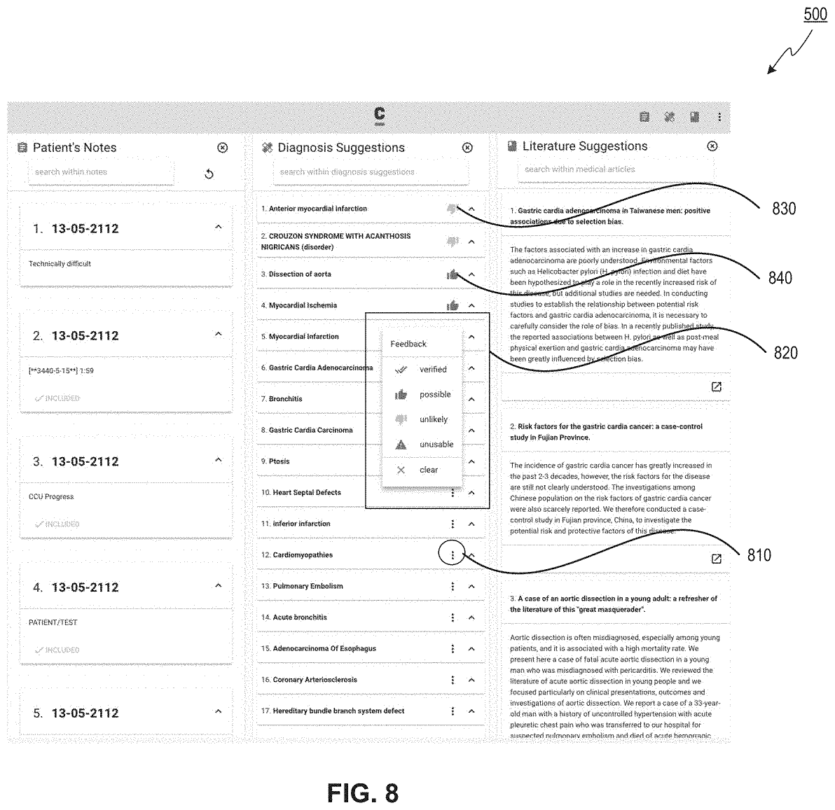

FIG. 8 depicts an example of a graphical user interface (GUI) 500 of a disease diagnosis system depicting feedback providing mechanism, in accordance with one or more aspects of the disclosure. The third display component 530 (identified in FIG. 5) provides ellipses 810 for a particular diagnosis that can be clicked in order to access a flyout window 820 for providing feedback regarding that particular diagnosis. Using the flyout window 820, a user can select whether the potential diagnosis is a verified diagnosis, possible diagnosis, unlikely diagnosis, or unusable diagnosis. The user can also clear the diagnosis from the list of diagnosis shown for the combination of queries for the EHR. The feedback can be useful when multiple users (e.g., physicians, etc.) work with the system and are able to see feedback from other users. In some examples, the feedback may be saved in a database for further use by the system to refine calculation of the ranking of potential diagnosis or inclusion of the potential diagnosis in the result set using the combination of keywords from the queries. In the example, the first diagnosis is shown to have received a feedback of “unlikely” diagnosis via indicator 830, and the third diagnosis is shown to have received a feedback of “possible” diagnosis via indicator 840.

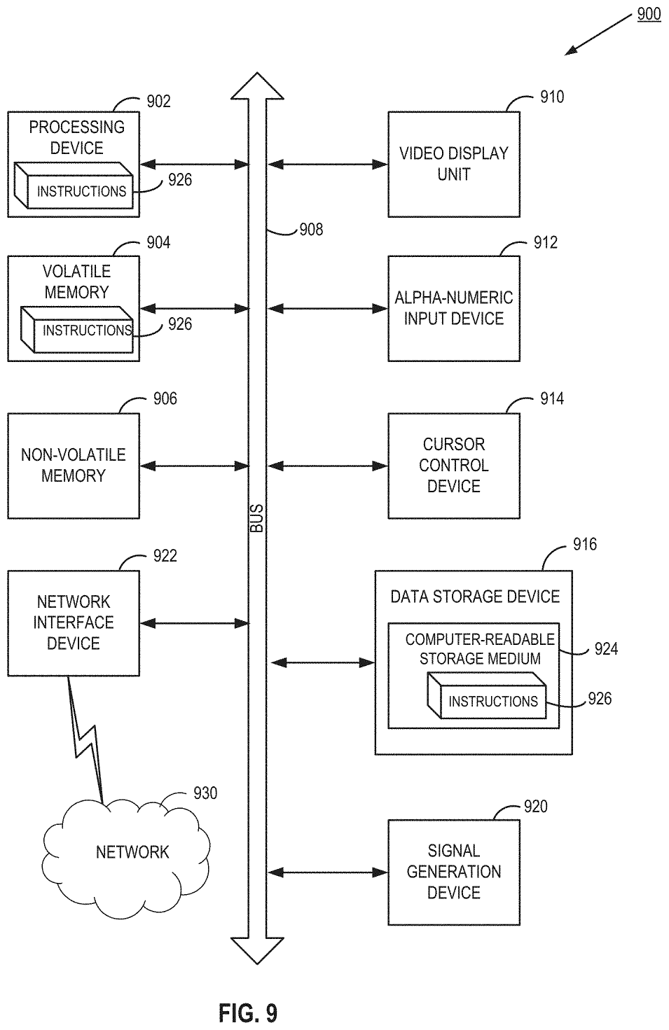

FIG. 9 depicts a block diagram of an example computer system 900 operating in accordance with one or more aspects of the disclosure. In various illustrative examples, computer system 900 may correspond to a computing device within system architecture 100 of FIG. 1. In certain implementations, computer system 900 may be connected (e.g., via a network 930, such as a Local Area Network (LAN), an intranet, an extranet, or the Internet) to other computer systems. Computer system 900 may operate in the capacity of a server or a client computer in a client-server environment, or as a peer computer in a peer-to-peer or distributed network environment. Computer system 900 may be provided by a personal computer (PC), a tablet PC, a set-top box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 900 may include a processing device 902, a volatile memory 904 (e.g., random access memory (RAM)), a non-volatile memory 906 (e.g., read-only memory (ROM) or electrically-erasable programmable ROM (EEPROM)), and a data storage device 916, which may communicate with each other via a bus 908.

Processing device 902 may be provided by one or more processors such as a general purpose processor (such as, for example, a complex instruction set computing (CISC) microprocessor, a reduced instruction set computing (RISC) microprocessor, a very long instruction word (VLIW) microprocessor, a microprocessor implementing other types of instruction sets, or a microprocessor implementing a combination of types of instruction sets) or a specialized processor (such as, for example, an application specific integrated circuit (ASIC), a field programmable gate array (FPGA), a digital signal processor (DSP), or a network processor).

Computer system 900 may further include a network interface device 922.

Computer system 900 also may include a video display unit 910 (e.g., an LCD, a touch enabled display unit, etc.), an alphanumeric input device 912 (e.g., a keyboard), a cursor control device 914 (e.g., a mouse), and a signal generation device 920.

Data storage device 916 may include a non-transitory computer-readable storage medium 924 on which may store instructions 926 encoding any one or more of the methods or functions described herein, including instructions for implementing method 200 of FIG. 2.

Instructions 926 may also reside, completely or partially, within volatile memory 904 and/or within processing device 902 during execution thereof by computer system 900, hence, volatile memory 904 and processing device 902 may also constitute machine-readable storage media.

While computer-readable storage medium 924 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by component modules or functional circuitry within hardware devices. Further, the methods, components, and features may be implemented in any combination of hardware devices and computer program components, or in computer programs.

Unless specifically stated otherwise, terms such as “generating,” “providing,” “training,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. Also, the terms “first,” “second,” “third,” “fourth,” etc. as used herein are meant as labels to distinguish among different elements and may not have an ordinal meaning according to their numerical designation.

Examples described herein also relate to an apparatus for performing the methods described herein. This apparatus may be specially constructed for performing the methods described herein, or it may comprise a general purpose computer system selectively programmed by a computer program stored in the computer system. Such a computer program may be stored in a computer-readable tangible storage medium.

The methods and illustrative examples described herein are not inherently related to any particular computer or other apparatus. Various general purpose systems may be used in accordance with the teachings described herein, or it may prove convenient to construct more specialized apparatus to perform method 200 and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

What is claimed is:

1. A method comprising:
accessing data associated with a patient;
dividing the data into one or more queries, wherein each of the one or more queries is associated with one or more keywords;
generating, for each of the one or more queries, a plurality of literatures based on the one or more keywords;
merging a plurality of terms extracted from each of the plurality of literatures for each of the one or more queries into a combined list of terms; and
providing, by a processing device, one or more potential diagnoses based on the combined list of terms.

2. The method of claim 1, wherein the data comprises one or more of:
a health record; or
a user input.

3. The method of claim 1, further comprising:
preprocessing the one or more queries to remove an uninformative keyword from the one or more keywords.

4. The method of claim 1, further comprising:
calculating a rank for each of the plurality of literatures for each of the one or more queries based on a relevance score associated with each of the plurality of literatures.

5. The method of claim 4, wherein the relevance score is calculated based on a number of matches between the plurality of terms from each of the plurality of literatures and the one or more keywords for each of the queries.

6. The method of claim 4, wherein the rank is calculated using a Bayesian language model with Dirichlet priors.

7. The method of claim 4, wherein the plurality of literatures comprise a specified number of literatures.

8. The method of claim 7, wherein the specified number of literatures is determined based on a difference between the relevance score of two consecutively ranked literatures having a largest value.

9. The method of claim 4, wherein the plurality of terms is determined based on an overall score calculated for each of the plurality of terms.

10. The method of claim 9, wherein the overall score is calculated based on a term score indicating a term frequency-inverse document frequency for a particular term of the plurality of terms and the relevance score associated with a particular literature corresponding to the particular term.

11. The method of claim 1, wherein the plurality of terms is determined by identifying, using a medical language library, a set of terms to remove from an initial set of extracted terms from each of the plurality of literatures.

12. The method of claim 1, further comprising:
grouping one or more synonymous terms of the plurality of terms under a unique identifier corresponding to a potential diagnosis of the one or more potential diagnoses.

13. A method comprising:
causing for display, by a processing device, a graphical user interface comprising:
a first display component graphically depicting a health record associated with a patient, wherein the health record is divided into one or more sections, each of the one or more sections corresponding to a distinct medical episode;
a second display component providing a plurality of literatures associated with the health record, wherein the plurality of literatures is generated based on one or more keywords associated with the health record; and
a third display component providing one or more potential diagnoses based on terms extracted from each of the plurality of literatures associated with the health record.

14. The method of claim 13, further comprising:
detecting a change in the health record; and
responsive to the change in the health record,
updating the first display component to depict the changed health record;
updating the second display component to depict an updated plurality of literatures associated with the changed health record; and
updating the third display component to provide an updated one or more potential diagnoses based on the changed health record.

15. The method of claim 13, wherein the health record comprises data input by a user.

16. A system comprising:
a memory; and
a processing device coupled with the memory to:
receive one or more user input associated with a patient;
divide the one or more user input into one or more queries, wherein each of the one or more queries is associated with one or more keywords;
generate, for each of the one or more queries, a plurality of literatures based on the one or more keywords;
merge a plurality of terms extracted from each of the plurality of literatures for each of the one or more queries into a combined list of terms; and
provide one or more potential diagnoses based on the combined list of terms.

17. The system of claim 16, wherein the processing device is further to:
calculate a rank for each of the plurality of literatures for each of the one or more queries based on a relevance score associated with each of the plurality of literatures.

18. The system of claim 17, wherein the relevance score is calculated based on a number of matches between the plurality of terms from each of the plurality of literatures and the one or more keywords for each of the queries.

19. A non-transitory computer readable storage medium encoding instructions thereon that, in response to execution by one or more processing devices, cause the processing device to perform operations comprising:
accessing a health record associated with a patient;
dividing the health record into one or more queries, wherein each of the one or more queries is associated with one or more keywords;
generating, for each of the one or more queries, a plurality of literatures based on the one or more keywords;
merging a plurality of terms extracted from each of the plurality of literatures for each of the one or more queries into a combined list of terms; and
providing one or more potential diagnoses based on the combined list of terms.

20. The non-transitory computer readable storage medium of claim 19, wherein the plurality of literatures comprise a specified number of literatures.
